SEQUENCE LISTING (4 sequences)

SEQ ID NO: 1 - DNA, 20 bp, Artificial Sequence
  The sequence is synthetized.
tgctccagtgcccgtagatc

SEQ ID NO: 2 - DNA, 20 bp, Artificial Sequence
  The sequence is synthetized.
gatctacgggcactggagca

SEQ ID NO: 3 - DNA, 20 bp, Artificial Sequence
  The sequence is synthetized.
cgcttccgcggcccgttcaa

SEQ ID NO: 4 - DNA, 20 bp, Artificial Sequence
  The sequence is synthetized.
ttgaacgggccgcggaagcg

(19) United States
(12) Patent Application Publication
(10) Pub. No.: US 2024/0423945 A1
(43) Pub. Date: Dec. 26, 2024

(54) USE OF FERROPTOSIS INHIBITOR IN PREPARATION OF DRUG FOR TREATING GASTRITIS

(71) Applicant: CHILDREN'S HOSPITAL OF CHONGQING MEDICAL UNIVERSITY, Chongqing (CN)

(72) Inventors: Yi HUANG, Chongqing (CN); Meihua YANG, Chongqing (CN); Jinmei FENG, Chongqing (CN); Xin ZENG, Chongqing (CN); Xiaohui XU, Chongqing (CN); Jinbao ZANG, Chongqing (CN)

(73) Assignee: CHILDREN'S HOSPITAL OF CHONGQING MEDICAL UNIVERSITY, Chongqing (CN)

(21) Appl. No.: 18/274,487

(22) PCT Filed: May 17, 2022
(86) PCT No.: PCT/CN2022/093212

§ 371 Date: Jun. 20, 2024

(30) Foreign Application Priority Data
May 9, 2022 (CN) 202210497705.3

(51) Int. Cl. A61K 31/245; A61P 1/04

(52) U.S. Cl. CPC A61K 31/245; A61P 1/04

(57) ABSTRACT

A use of a ferroptosis inhibitor in preparation of a drug for treating gastritis is provided. The ferroptosis inhibitor includes ferrostatin-1 or a derivative, isomer, or pharmaceutically acceptable salt thereof as an active ingredient, and further includes a pharmaceutically acceptable carrier, an excipient, a diluent, an adjuvant, a vehicle, or a combination thereof. The present disclosure discovers that the ferroptosis inhibitor ferrostatin-1 can effectively inhibit a pathological process of chronic atrophic gastritis (CAG), and in vivo and in vitro experiments confirm that the ferroptosis inhibitor ferrostatin-1 can be used for prevention and treatment of CAG, which can provide a new idea for development and innovation of drugs for treating CAG.

### CROSS REFERENCE TO THE RELATED APPLICATIONS

This application is the national phase entry of International Application No. PCT/CN2022/093212, filed on May 17, 2022, which is based upon and claims priority to Chinese Patent Application No. 202210497705.3, filed on May 9, 2022, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBCD141-PKG_Sequence_Listing.txt, created on Jul. 13, 2023, and is 1,161 bytes in size.

### TECHNICAL FIELD

The present disclosure belongs to the technical field of medicine, and specifically relates to a use of a ferroptosis inhibitor in preparation of a drug for treating gastritis.

### BACKGROUND

Chronic atrophic gastritis (CAG) is a precancerous lesion of gastric cancer (GC) with “inflammation→atrophy→metaplasia→dysplasia” as prominent pathological features, and is closely related to the occurrence of intestinal GC. Metaplasia, as an important node of a pathological process of CAG, is key to the early prevention and control of intestinal GC, but the formation mechanism of metaplasia is not completely clear. Therefore, the research on the formation etiology and mechanism of metaplasia is of great significance for the early prevention and control of intestinal GC.

Chinese patent CN113940309A discloses a method for efficiently simulating a CAG lesion with high simulation degree, excellent stability and durability, and specific phenotype: a gene-edited mouse GRIM-19^{flox/flox} is hybridized with a gene-edited mouse ATP4b-Cre to obtain a gastric mucosa-specific parietal cell GRIM-19-knockout mouse strain (GRIM-19^{-/-}/ATP4b-Cre, SPF level), and the gene-knockout mouse strain has an early lesion of CAG intestinal metaplasia (namely, spontaneous spasmolytic polypeptide expression metaplasia (SPEM)) at an age of 8 months, and can undergo stable expression without repetition, which can be used as a mouse CAG pathological model for research.

Ferroptosis, also known as iron-dependent death, is a new regulated cell death form that is different from apoptosis, necrosis, and autophagy and is closely related to a variety of diseases such as neurodegeneration, tumor, ischemia-reperfusion injury (IRI), kidney damage, and liver fibrosis. Ferroptosis is typically characterized by intracellular lipid peroxide accumulation, where excess intracellular Fe²⁺ leads to antioxidant glutathione (GSH) depletion and glutathione peroxidase 4 (GPX4) inactivation through a Fenton reaction, and thus induces lipid peroxide accumulation, thereby causing ferroptosis.

Ferrostatin-1, a currently recognized ferroptosis inhibitor, is a synthetic antioxidant that can prevent membrane lipids from being damaged through a reduction mechanism to inhibit cell death. Chinese patent CN111529518A discloses that the ferroptosis inhibitor ferrostatin-1 can significantly improve lung injury and cell damage caused by drowning in mice, and has a significant protective effect for lung damage caused by drowning. Chinese patent CN113440509A discloses that the ferroptosis inhibitor ferrostatin-1 can significantly increase the content of intracellular GPX4, significantly inhibit the production of reactive oxygen species (ROS) in osteoblasts stimulated by wear particles, and reduce the ferroptosis of osteoblasts; and an in vivo experiment shows that the intraperitoneal injection of ferrostatin-1 has a significant inhibitory effect on osteolysis induced by wear particles. However, there are currently no reports on a role of ferroptosis inhibitors and derivatives thereof in CAG.

### SUMMARY

In view of the above deficiencies in the prior art, the present disclosure provides a new use of a ferroptosis inhibitor in preparation of a drug for treating gastritis. The present disclosure discovers for the first time that the GRIM-19 deletion can induce the slowed proliferation and reduced vitality of human gastric mucosal epithelial cells through a mechanism of ferroptosis, rather than apoptosis, necrosis, autophagy, and other death forms.

An objective of the present disclosure is to provide a ferroptosis inhibitor ferrostatin-1 as a therapeutic target for CAG characterized by GRIM-19 deletion, where the ferrostatin-1 can increase a level of GPX4 in a gastric mucosal tissue and specifically catalyze the conversion of lipid peroxides by GSH into steroid selenoprotein to remove toxic products in a ferroptosis process, thereby further inhibiting the occurrence of ferroptosis. In addition, the ferrostatin-1 can significantly reduce the infiltration of inflammatory cells and inflammatory factors in a gastric mucosal tissue caused by the deletion of GRIM-19 in gastric mucosal parietal cells of mice, reduce the SPEM index, and effectively inhibit the pathological process of CAG, which provides a new idea for development and innovation of drugs for treating CAG.

To achieve the above objective, the present disclosure adopts the following technical solutions to solve the technical problems of the present disclosure:

A use of a ferroptosis inhibitor in preparation of a drug for treating gastritis is provided.

Further, the ferroptosis inhibitor includes ferrostatin-1 as an active ingredient, and further includes a pharmaceutically acceptable carrier, an excipient, a diluent, an adjuvant, a vehicle, or a combination thereof.

Further, the active ingredient in the ferroptosis inhibitor is a derivative, an isomer, or a pharmaceutically acceptable salt of the ferrostatin-1.

Further, the gastritis is CAG.

Further, the CAG refers to CAG induced by a defect in a GRIM-19 gene.

Further, the ferroptosis inhibitor is able to inhibit expression of a SPEM gene.

Further, the ferroptosis inhibitor can increase a level of the ferroptosis marker protein GPX4 in a CAG model.

Further, the ferroptosis inhibitor can reduce expression of inflammatory factors such as L-6, TNF-α, VEGF, GM-CSF, IL-1β, and IL-33 in a mouse gastric mucosal tissue of a CAG model.

Further, the ferroptosis inhibitor can protect a gastric mucosal tissue.

Further, the drug is a tablet, a suppository, an injection, or a capsule.

Further, the drug is an injection, and is administered through intraperitoneal injection at a dose of 1 mg/kg to 1.5 mg/kg.

The present disclosure has the following beneficial effects:

The present disclosure discovers that the in vivo injection of the ferroptosis inhibitor ferrostatin-1 can significantly increase a level of GPX4 in a mouse gastric mucosal tissue and inhibit a pathological process of ferroptosis of gastric mucosal parietal cells to effectively reduce the expression of inflammatory factors such as L-6, TNF-α, VEGF, GM-CSF, IL-1β, and IL-33 in the mouse gastric mucosal tissue, significantly reduce the SPEM index, and effectively inhibit a pathological process of CAG, and thus the ferroptosis inhibitor can be used in treatment of CAG. The in vitro treatment of GES-1-134 cells by ferrostatin-1 can increase a level of GPX4 in cells and inhibit a pathological process of ferroptosis, thereby enhancing the viability of cells. In vivo and in vitro experiments confirm that the ferroptosis inhibitor ferrostatin-1 can be used for treatment of CAG, which can provide a new idea for development and innovation of drugs for treating CAG.

### BRIEF DESCRIPTION OF THE DRAWINGS

FIGS. 1A-1B show the construction of a monoclone of a GRIM-19-knockout human gastric mucosal epithelial cell GES-1 (GES-1-134), where FIG. 1A shows the GFP positive detection results of the monoclone by flow cytometry (FCM); and FIG. 1B shows the expression analysis results of the GRIM-19 protein in the GES-1-134 cell detected by Western blot (WB).

FIG. 2 shows the detection results of viability of a GES-1-134 cell.

FIGS. 3A-3B show the detection results of apoptosis, necrosis, and autophagy of a GES-1-134 cell, where FIG. 3A shows the apoptosis and necrosis of a GES-1-134 cell detected by FCM; and FIG. 3B shows the analysis results of expression of the autophagy markers LC3I/LC3II in the GES-1-134 cell detected by WB.

FIGS. 4A-4B show the detection results of ferroptosis marker proteins in a GES-1-134 cell and a GRIM-19^{-/-} mouse gastric mucosal tissue, where FIG. 4A shows the expression levels of ferroptosis marker proteins (COX2, GPX4, FTH1, and TFR) in the GES-1-134 cell detected by WB; and FIG. 4B shows the expression levels of ferroptosis marker proteins (COX2, GPX4, FTH1, and TFR) in the GRIM-19^{-/-} mouse gastric mucosal tissue detected by WB.

FIGS. 5A-5B show transmission electron microscopy (TEM) images of a GES-1-134 cell and a GRIM-19^{-/-} mouse gastric mucosal tissue, where FIG. 5A shows the TEM analysis of a mitochondrial phenotype of the GES-1-134 cell; and FIG. 5B shows the TEM analysis of a mitochondrial phenotype of the GRIM-19^{-/-} mouse gastric mucosal tissue.

FIGS. 6A-6B show the detection results of iron ions in a GES-1-134 cell and a GRIM-19^{-/-} mouse gastric mucosal tissue, where FIG. 6A shows the detection results of a Fe²⁺ content in the GES-1-134 cell; and FIG. 6B shows the Prussian blue (PB) staining results of the GRIM-19^{-/-} mouse gastric mucosal tissue (*<0.05).

FIG. 7 shows the detection results of lipid oxidation (MDA) of a GES-1-134 cell (***<0.001).

FIG. 8 shows the detection results of viability of a GES-1-134 cell intervened with the ferroptosis inhibitor ferrostatin-1 (***<0.001).

FIGS. 9A-9B show the detection results of expression of ferroptosis marker proteins in a GES-1-134 cell and a GRIM-19^{-/-} mouse gastric mucosal tissue that each are intervened with the ferroptosis inhibitor ferrostatin-1, where FIG. 9A shows the WB detection results of expression levels of ferroptosis marker proteins (COX2, GPX4, FTH1, and TFR) in the GES-1-134 cell treated with the ferroptosis inhibitor ferrostatin-1 (0 μM, 5 μM, and 10 μM); and FIG. 9B shows the WB detection results of expression levels of ferroptosis marker proteins (COX2, GPX4, FTH1, and TFR) in the GRIM-19^{-/-} mouse gastric mucosal tissue intervened with the ferroptosis inhibitor ferrostatin-1.

FIGS. 10A-10B show the detection results of expression of inflammatory factors in a GES-1-134 cell and a GRIM-19^{-/-} mouse gastric mucosal tissue that each are intervened with the ferroptosis inhibitor ferrostatin-1, where FIG. 10A shows the WB detection results of expression levels of IL-6, TNF-α, VEGF, GM-CSF, IL-1β, and IL-33 proteins in the GES-1-134 cell intervened with ferrostatin-1; and FIG. 10B shows the WB detection results of expression levels of IL-6, TNF-α, VEGF, GM-CSF, IL-1β, and IL-33 proteins in the GRIM-19^{-/-} mouse gastric mucosal tissue intervened with ferrostatin-1.

FIGS. 11A-11B show the detection and analysis results of a pathological process of SPEM in a GRIM-19^{-/-} mouse gastric mucosal tissue intervened with the ferroptosis inhibitor ferrostatin-1, where FIG. 11A shows the WB detection results of SPEM marker proteins TFF2, Mist1, Clusterin-1, HE4, and MUC6 in the gastric mucosal tissue; and FIG. 11B shows the GIF, GSII, and Mist triple immunofluorescence assay (TIFA) results (Bar: 50 μM) (***<0.001).

### DETAILED DESCRIPTION OF THE EMBODIMENTS

The specific implementations of the present disclosure are described below to facilitate those skilled in the art to understand the present disclosure, but it should be known that the present disclosure is not limited to the scope of the specific implementations. Various obvious changes made by those of ordinary skill in the art within the spirit and scope of the present disclosure defined by the appended claims should fall within the protection scope of the present disclosure.

### Example 1 In Vitro Construction of a GES-1-134 Cell Model

### 1. Construction of a Lentiviral Vector System

In the present disclosure, a CRISPR/CAS9 single-vector lentiviral system from Shanghai Genechem Co., Ltd. was adopted.
(1) A frame structure of a lentivirus GV393 was U6-sgRNA-EIF1a-Cas9-FLAG-P2A-EGFP.
(2) Target gene information:

| Target gene name | Gene species | Gene ID | GenBank ID |
|---|---|---|---|
| NDUFA13 (GRIM-19) | Human | 51079 | NM_015965 |

(3) Target sgRNA primer sequences:

| | | | |
|---|---|---|---|
| NDUFA-13-sgRNA-134-F | CACCg | TGCTCCAGTGCCCGTAGATC, | / |
| | | as shown in SEQ ID NO: 1 | |
| NDUFA-13-sgRNA-134-R | AAAC | GATCTACGGGCACTGGAGCA, | C |
| | | as shown in SEQ ID NO: 2 | |
| NC-sgRNA-F | CACCg | CGCTTCCGCGGCCCGTTCAA, | / |
| | | as shown in SEQ ID NO: 3 | |
| NC-sgRNA-R | AAAC | TTGAACGGGCCGCGGAAGCG, | C |
| | | as shown in SEQ ID NO: 4 | |

### 2. GES-1 (Human Normal Gastric Mucosal Epithelial Cell) Infection

(1) GES-1 cells were inoculated into a 96-well plate at 1,000 cells/well.
(2) Infection was conducted when a cell growth density reached about 30% to 40%.
A. Before the infection, a HitransG P infection-enhancing solution was diluted with a complete medium according to 1:25 for later use.
B. A virus usage was calculated according to multiplicity of infection (MOI)=50.
C. The cell was cultivated in a 37° C., 5% CO2 incubator, the cell morphology was observed about 8 h to 16 h after infection, and a medium was changed.
D. 72 h after infection, an infection effect was observed under a microscope.

### 3. Cell Sorting by FCM

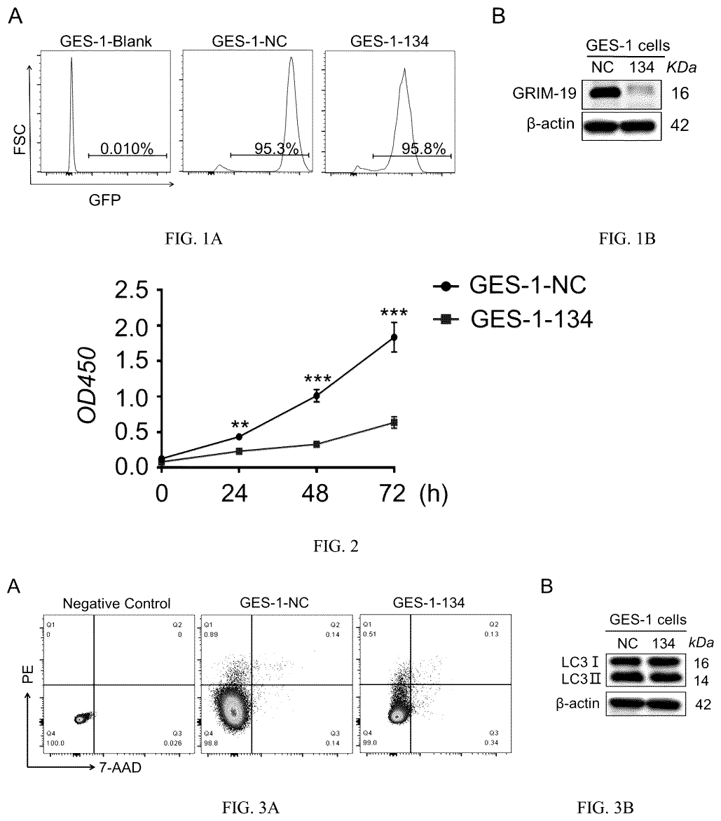

GFP-positive cells were sorted by FCM, then diluted into single cells, and then subjected to monoclonal proliferation to obtain a high-purity GFP-positive cell, as shown in FIG. 1A.

4. The expression of GRIM-19 was analyzed by WB, and a specific process was as follows:
(1) The cells were subjected to lysis with an RIPA lysis buffer (Beyotime) on ice for 30 min or more.
(2) A resulting lysate was centrifuged at 12,000 rpm for 10 min, and a resulting supernatant was collected.
(3) A corresponding volume of a sodium dodecyl sulfate-polyacrylamide gel electrophoresis (SDS-PAGE) protein loading buffer was added, and a resulting mixture was incubated in a metal bath at 100° C. for 10 min.
(4) SDS-PAGE was conducted.
(5) Blocking was conducted at room temperature for 30 min.
(6) A primary antibody was added, and a resulting mixture was incubated overnight at 4° C. on a shaker.
(7) A membrane was washed with TBST three times for 10 min each time.
(8) A secondary antibody was added, and a resulting mixture was incubated for 1 h at room temperature on a shaker.
(9) A membrane was washed with TBST three times for 10 min each time.
(10) Development was conducted.

As shown in FIG. 1B, a level of the GRIM-19 protein in GES-1-134 was significantly reduced compared with the control, indicating that a human normal gastric mucosal cell GES-1 GRIM-19-knockout model was successfully constructed.

### Example 2 Analysis of Proliferation Viability of a GES-1-134 Cell

A CCK-8 kit (Solarbio) was used to analyze the proliferation viability changes of GES-1-NC and GES-1-134 cells, and a specific process was as follows:
(1) The cells were inoculated into a 96-well plate at 5,000 cells/well.
(2) The cells were cultivated at 37° C. and 5% CO2 for 24 h, 48 h, and 72 h.
(3) 10 μL of a CCK-8 solution was added to each well during detection.
(4) The plate was incubated in an incubator for 1 h.
(5) A microplate reader was used to determine the absorbance at 450 nm.

Results were shown in FIG. 2. The proliferation rate and viability of GES-1-134 both were reduced compared with the control.

### Example 3 Apoptosis Analysis of a GES-1-134 Cell

An apoptosis kit was used to analyze the apoptosis of GES-1-134, and a specific process was as follows:
(1) 5×10⁵ cells were collected.
(2) The cells were rinsed with PBS twice (2,000 rpm/5 min).
(3) A mixture of binding buffer and 7-AAD in a ratio of 10:1 was added, 50 μL of a stain solution was added, and a resulting mixture was thoroughly mixed and subjected to a reaction in the dark for 15 min.
(4) A mixture of binding buffer and Annexin V-PE in a ratio of 450:1 was added, 450 μL of a stain solution was added, and a resulting mixture was thoroughly mixed and subjected to a reaction in the dark for 15 min.
(5) FCM analysis was conducted within 1 h.

Results were shown in FIG. 3A. There was no significant difference in apoptosis between the control GES-1-NC and the GES-1-134, indicating that the reduced proliferation rate and viability of gastric mucosal epithelial cells induced by the deletion of GRIM-19 was not achieved by apoptosis.

### Example 4 Autophagy Analysis of a GES-1-134 Cell

The expression levels of autophagy marker proteins LC3I and LC3II in GES-1-134 were detected by WB. As shown in FIG. 3B, compared with the control group, the expression levels of autophagy markers LC3I and LC3II in GES-1-134 were not increased, indicating that the reduced proliferation rate and viability of gastric mucosal epithelial cells induced by the deletion of GRIM-19 was not achieved by autophagy.

### Example 5 Analysis of Expression of Ferroptosis Markers in a GES-1-134 Cell and a GRIM-19^{-/-} Mouse Gastric Mucosal Tissue

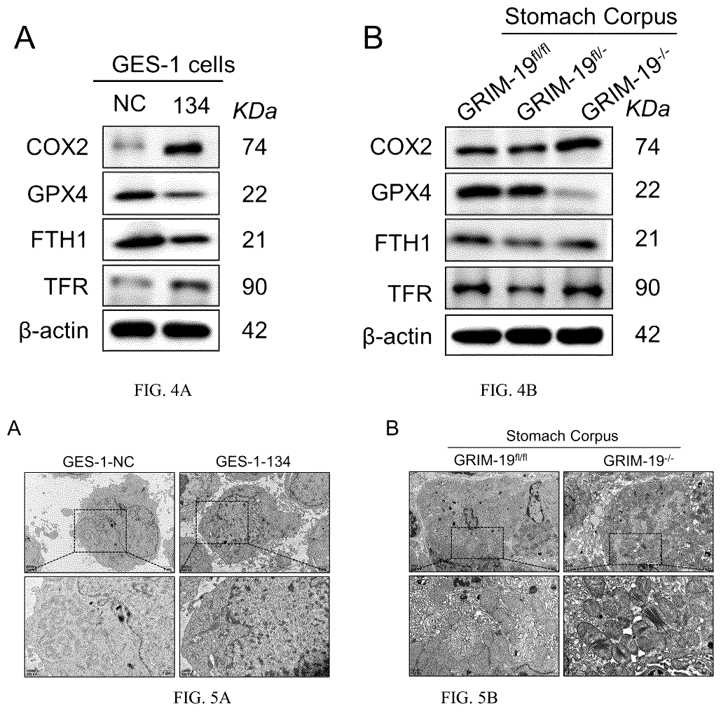

WB was used to detect the expression of ferroptosis-associated marker proteins in the GES-1-134 cell and the GRIM-19^{-/-} mouse gastric mucosal tissue. Results were shown in FIG. 4A.

Compared with the GES-1-NC cell, in the GES-1-134 cell, the expression levels of GPX4 and ferritin (FTH1) were decreased, and the expression levels of cyclooxygenase (COX-2) and transferrin receptor (TFR) were increased, indicating that, after GRIM-19 was knocked out from human normal gastric mucosal epithelial cells, the Fe²⁺ was over-loaded and the lipid peroxide clearance was weakened, such that the ferroptosis occurred. As shown in FIG. 4B, compared with the control group, the expression of ferroptosis marker proteins in the GRIM-19^{fl/-} and GRIM-19^{-/-} mouse gastric mucosal tissues showed the same trend, indicating that the deletion of GRIM-19 in mouse gastric mucosal parietal cells induced a parietal cell damage through a mechanism of ferroptosis.

### Example 6 Electron Microscopy (EM) Analysis of a GES-1-134 Cell and a GRIM-19^{-/-} Mouse Gastric Mucosal Tissue

A TEM analysis technique was used to detect the changes of mitochondrial structures of a GRIM-19^{-/-} mouse gastric mucosal tissue and a GES-1-134 cell, and a specific process was as follows:
(1) A fresh tissue was cut to a size of 1 mm³, an EM fixing solution was immediately added, and a resulting mixture was stored in a refrigerator at 4° C. (which could be stored for 3 to 6 months) and sent to an EM room for testing on a specified date.
(2) 1×10⁶ of cells were digested and then centrifuged at 1,200 rpm for 10 min to obtain a tight cell mass at a bottom of an Ep tube, a resulting supernatant was completely removed, a fixing solution was gently added along a wall of the tube (the cell mass should not be destroyed), and the tube was stored in a refrigerator at 4° C. (which could be stored for 3 to 6 months) and sent to an EM room for testing on a specified date.

TEM analysis results were shown in FIG. 5A. Compared with GES-1-NC, the structure and number of mitochondria in the GES-1-134 cell were significantly changed: decreased number, increased membrane density, and decreased crests, which were consistent with the morphological characteristics of ferroptosis. As shown in FIG. 5B, compared with control mouse, there are similar changes in the mitochondrial structure of the GRIM-19^{-/-} mouse gastric mucosal tissue: increased mitochondrial membrane density, decreased crests, and structural disorders in tabular crests. The above in vivo and in vitro TEM analysis confirmed that the deletion of GRIM-19 induced the morphological changes of ferroptosis in mitochondria of the human gastric mucosal epithelial cell and the mouse gastric mucosal parietal cell.

### Example 7 Detection Analysis of Ferrous Ions in a GES-1-134 Cell

The excess intracellular Fe²⁺ may lead to antioxidant GSH depletion and GPX4 inactivation through a Fenton reaction, and thus induce lipid peroxide accumulation, thereby causing ferroptosis. A Fe²⁺ content in a GES-1-34 cell was detected by a ferrous ion colorimetric kit (Elabscience), and a specific process was as follows:
(1) 4 to 6×10⁶ cells were collected.
(2) A reagent 1 was added to conduct homogenization.
(3) A homogenate was centrifuged at 1,000 g for 10 min, and a resulting supernatant was taken for later use.
(4) A reagent 2 was added, and a resulting mixture was incubated at 37° C. for 10 min.
(5) The mixture was centrifuged at 12,000 g for 10 min, and a resulting supernatant was taken for later use.
(6) A microplate reader was used to determine an OD value at 532 nm.

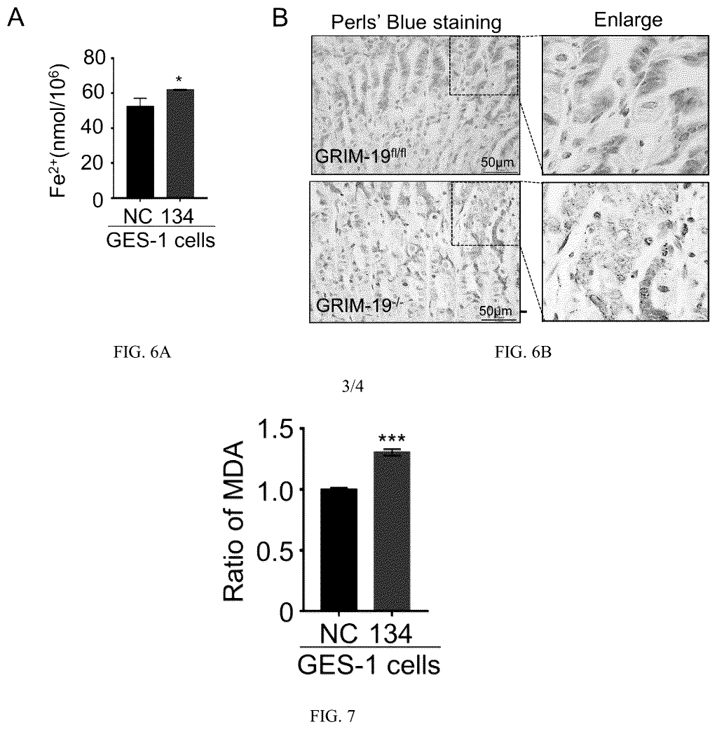

Results were shown in FIG. 6A. A content of Fe²⁺ in the GES-1-134 cell was increased, indicating that the knockout of GRIM-19 induced the increased Fe²⁺ in the gastric mucosal epithelial cell, and Fe²⁺ could promote the occurrence of ferroptosis through a Fenton reaction.

### Example 8 PB Detection Analysis of an Iron Ion in a GRIM-19^{-/-} Mouse Gastric Mucosal Tissue

A PB kit (Solarbio) was used to detect the expression of the iron ion in the mouse gastric mucosal tissue, and a specific process was as follows:
(1) Tissue fixation;
(2) conventional dehydration and embedding;
(3) sectioning and baking;
(4) drop-staining with a Perls stain working solution for 15 min;
(5) washing with distilled water for 5 min;
(6) drop-staining with a nuclear fast red solution for 10 min;
(7) washing with tap water;
(8) conventional dehydration and permeabilization; and
(9) mounting with a neutral resin.

Results were shown in FIG. 6B. Compared with the control group, a content of the iron ion in the GRIM-19^{-/-} mouse gastric mucosal tissue was significantly increased, indicating that the iron overload in the gastric mucosal tissue was further aggravated to cause the occurrence of ferroptosis.

### Example 9 Detection Analysis of Lipid Oxidation (MDA) in a GES-1-134 Cell

Lipid oxidation occurs when an oxidative stress occurs in an animal or plant cell. A lipid oxidation (MDA) detection kit (Beyotime) was used to analyze lipid oxidation levels of GES-1-134 and a mouse gastric mucosal tissue, and a specific process was as follows:
(1) 1×10⁶ cells were collected and subjected to lysis.
(2) A lysate was centrifuged at 10,000 g for 10 min, and a resulting supernatant was collected.
(3) 0.1 mL of an MDA working solution was added, and a resulting mixture was incubated in a 100° C. metal bath for 15 min.
(4) The mixture was centrifuged at 1,000 g for 10 min, and a resulting supernatant was collected.
(5) A microplate reader was used to determine the absorbance at 532 nm.

Results were shown in FIG. 7. Compared with the control group, the MDA of the GES-1-134 cell was increased significantly, indicating that the knockout of GRIM-19 caused an increase of MDA in the human gastric mucosal epithelial cell, which further promoted the occurrence of ferroptosis.

### Example 10 Assay Analysis of Proliferation Viability of a GES-1-134 Cell Intervened with Ferrostatin-1

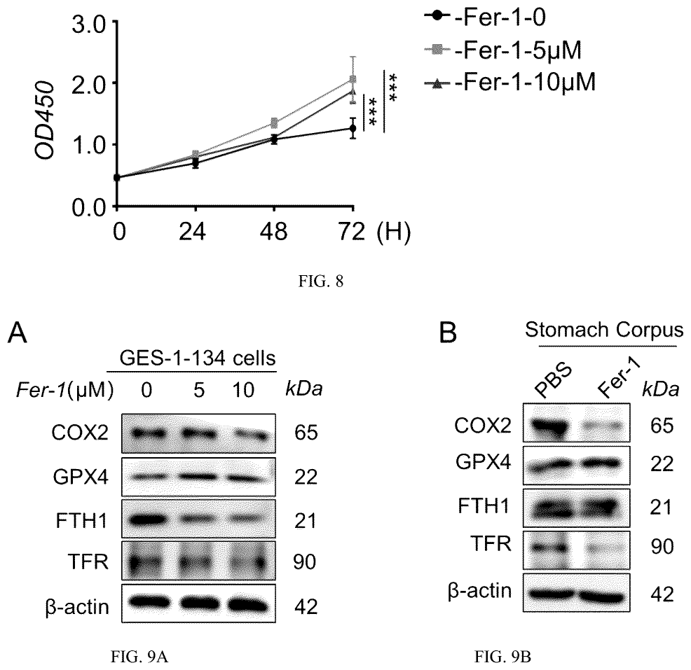

A CCK-8 kit was used to analyze a change of proliferation viability of the GES-1-134 cell intervened with the ferroptosis inhibitor ferrostatin-1. Results were shown in FIG. 8. After the GES-1-134 cell was intervened with the ferroptosis inhibitor ferrostatin-1, the proliferation ability was increased and the viability was recovered.

### Example 11 Analysis of Expression of Ferroptosis Markers in a GES-1-134 Cell and a GRIM-19^{-/-} Mouse that Each were Intervened with Ferrostatin-1

WB was used to analyze the expression of ferroptosis markers in the GES-1-134 cell and the GRIM-19^{-/-} mouse gastric mucosal tissue that each were intervened with ferrostatin-1.

As shown in FIG. 9A, after the GES-1-134 cell was treated with the ferroptosis inhibitor ferrostatin-1, the expression levels of ferroptosis marker proteins GPX4 and FTH1 were increased and the expression levels of ferroptosis marker proteins COX-2 and TFR were decreased, indicating that ferrostatin-1 effectively inhibited a pathological process of ferroptosis in the GRIM-19-knockout human gastric mucosal epithelial cell GES-1. In an in vivo experiment, ferrostatin-1 was injected into a GRIM-19-mouse, and as shown in FIG. 9B, the GPX4, FTH1, COX-2, and TFR showed the same change trend. The above in vitro and in vivo experiments confirmed that the ferroptosis inhibitor ferrostatin-1 effectively inhibited a pathological process of ferroptosis in the gastric mucosal cell induced by GRIM-19 deletion.

### Example 12 Analysis of Expression of Inflammatory Factors in a GES-1-134 Cell and a GRIM-19^{-/-} Mouse that Each were Intervened with Ferrostatin-1

WB was used to detect the expression of inflammatory factors in the GES-1-134 cell and GRIM-19^{-/-} mouse after being intervened with the ferroptosis inhibitor ferrostatin-1.

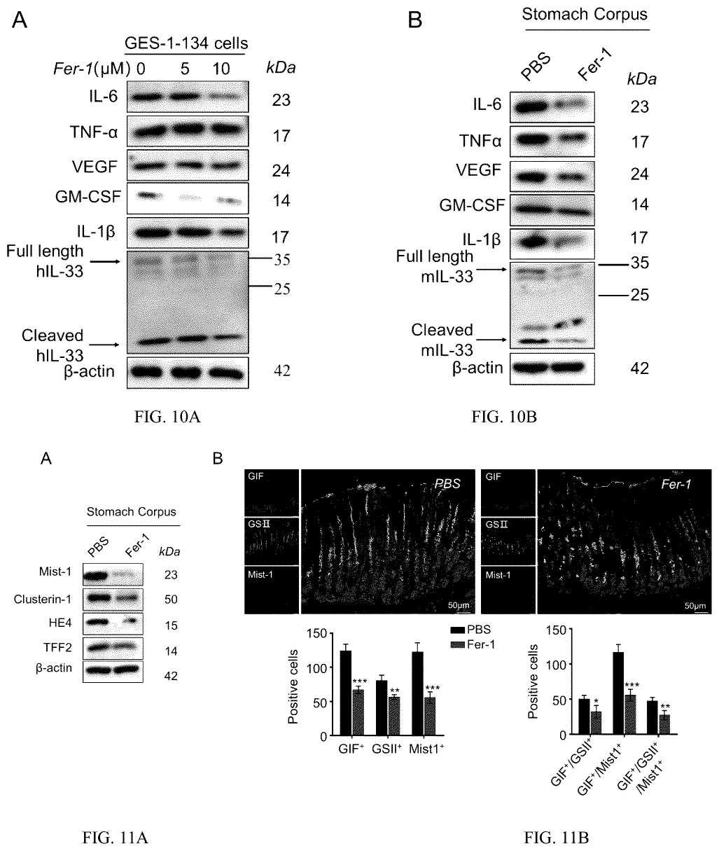

Results were shown in FIGS. 10A-10B. After the GES-1-134 cell was intervened with the ferroptosis inhibitor ferrostatin-1 in vitro, the expression levels of inflammatory factors IL-6, TNF-α, VEGF, GM-CSF, IL-1β, and IL-33 were significantly reduced. An in vivo experiment showed that, compared with the PBS injection control, the expression levels of inflammatory factors IL-6 and TNF-α in the Fer-1 group were also significantly reduced. The in vitro and in vivo experiments confirmed that the ferroptosis inhibitor ferrostatin-1 could reduce the expression of inflammatory factors in the GRIM-19-knockout human gastric mucosal epithelial cell and the gastric mucosal tissue of the parietal cell-specific GRIM-19-knockout mouse, effectively slow down an inflammatory response of the gastric mucosal tissue, and inhibit a pathological process of CAG.

### Example 13 Analysis of Expression of SPEM Markers in a GRIM-19^{-/-} Mouse Gastric Mucosal Tissue Intervened with Ferrostatin-1

WB was used to detect the expression of SPEM markers in the gastric mucosal tissue of the GRIM-19^{-/-} mouse intraperitoneally injected with the ferroptosis inhibitor ferrostatin-1.

Results were shown in FIG. 11A. Compared with the PBS injection control, the expression of TFF2, Mist1, Clusterin-1, HE4, and MUC6 in the ferroptosis inhibitor ferrostatin-1 group was significantly weakened, indicating that the ferroptosis inhibitor ferrostatin-1 could effectively inhibit a pathological process of SPEM of CAG in the GRIM-19^{-/-} mouse.

### Example 14 GIF/GSII/Mist1 Colocalization Analysis of SPEM Markers in a GRIM-19^{-/-} Mouse Gastric Mucosal Tissue Intervened with Ferrostatin-1

After a mouse was intraperitoneally injected with the ferroptosis inhibitor ferrostatin-1, an immunofluorescence technique was used to analyze the GIF, GSII, and Mist1 positive colocalization expression of SPEM markers in a gastric mucosal tissue, and a specific process was as follows:
(1) embedding a tissue with OCT;
(2) sectioning;
(3) fixing with 4% PFA;
(4) blocking with serum;
(5) incubating with a primary antibody overnight at 4° C.;
(6) rinsing with PBS 3 times;
(7) incubating with a secondary antibody at room temperature for 2 h;
(8) rinsing with PBS 3 times;
(9) counter-staining with DAPI for 10 min;
(10) rinsing with PBS 3 times; and
(11) mounting with an anti-fluorescence attenuation agent.

Results were shown in FIG. 11B. Compared with the PBS group, the number of positive cells was decreased significantly in the Fer-1, GIF⁺, GSII⁺, and Mist1⁺ groups, and the number of GIF⁺/GSII⁺ double-positive cells, the number of GIF⁺/Mist1+ double-positive cells, and the number of GIF⁺/GSII⁺/Mist1⁺ triple-positive cells were decreased, indicating that the ferroptosis inhibitor Ferrostatin-1 could effectively inhibit a pathological process of SPEM of CAG.

What is claimed is:

1. A method of using a ferroptosis inhibitor in a preparation of a drug for treating a gastritis.

2. The method according to claim 1, wherein the ferroptosis inhibitor comprises a ferrostatin-1 as an active ingredient, and further comprises a pharmaceutically acceptable carrier, an excipient, a diluent, an adjuvant, a vehicle, or a combination of the pharmaceutically acceptable carrier, the excipient, the diluent, the adjuvant, and the vehicle.

3. The method according to claim 2, wherein the active ingredient in the ferroptosis inhibitor is a derivative, an isomer, or a pharmaceutically acceptable salt of the ferrostatin-1.

4. The method according to claim 1, wherein the gastritis is a chronic atrophic gastritis (CAG).

5. The method according to claim 4, wherein the CAG is induced by a defect in a GRIM-19 gene.

6. The method according to claim 4, wherein the ferroptosis inhibitor is configured to inhibit an expression of a spasmolytic polypeptide expression metaplasia (SPEM) gene.

7. The method according to claim 4, wherein the drug is a tablet, a suppository, an injection, or a capsule.

8. The method according to claim 4, wherein the drug is an injection, and is administered through an intraperitoneal injection at a dose of 1 mg/kg to 1.5 mg/kg.

9. The method according to claim 2, wherein the gastritis is a CAG.

10. The method according to claim 3, wherein the gastritis is a CAG.
